(19) United States
(12) Patent Application Publication
(10) Pub. No.: US 2024/0246906 A1
(43) Pub. Date: Jul. 25, 2024

(54) A PROCESS FOR THE PREPARATION OF 2-NITRO-4-(METHYLSULFONYL)BENZOIC ACID

(71) Applicant: GHARDA CHEMICALS LIMITED, Dist.Thane, Maharashtra (IN)

(72) Inventors: Suchet Saran MATHUR, Dist.Thane, Maharashtra (IN); Shekhar Vishwanath SATHE, Dist.Thane, Maharashtra (IN); Mithilesh Kumar MAIRH, Dist.Thane, Maharashtra (IN); Kedar Parshuram KSHIRSAGAR, Dist.Thane, Maharashtra (IN); Vinodkumar Jethuram PRAJAPATI, Dist.Thane, Maharashtra (IN); Vikram Naresh SINGH, Dist.Thane, Maharashtra (IN)

(21) Appl. No.: 18/574,448

(22) PCT Filed: Jun. 23, 2022
(86) PCT No.: PCT/IB2022/055815

§ 371 Date: Dec. 27, 2023

(30) Foreign Application Priority Data
Jun. 29, 2021 (IN) 202121029249

(51) Int. Cl. C07C 317/14

(52) U.S. Cl. CPC C07C 317/14

(57) ABSTRACT

The present disclosure relates to a process for the preparation of 2-nitro-4-methylsulfonyl benzoic acid (NMSBA). The process comprises reacting 1-chloro-2-nitro-4-methyl sulfonyl benzene (chloro NMSB) with a compound of Formula (II) by using a base in a fluid medium at a first predetermined temperature to obtain a compound of Formula (III). The compound of Formula (III) is reacted with an oxidizing agent optionally in the presence of a catalyst under stirring to obtain a mixture. Air is optionally passed through the mixture. The mixture is heated at a second predetermined temperature for a predetermined time period to obtain a product mixture comprising 2-Nitro-4-(methyl sulfonyl) benzoic acid. The process of the present disclosure is simple, economical and environment-friendly.

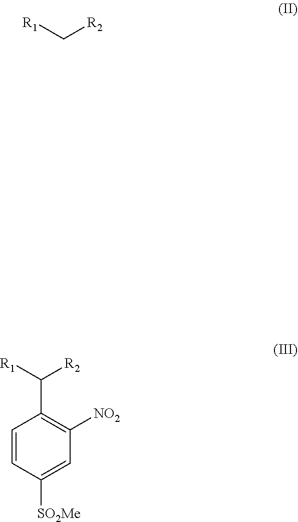

### FIELD

The present disclosure relates to a process for the preparation of 2-nitro-4-(methylsulfonyl)benzoic acid (NMSBA).

### BACKGROUND

The background information herein below relates to the present disclosure but is not necessarily prior art.

Benzoic acid and its substituted derivatives are used inter alia in agrochemicals and pharmaceuticals. The substituted benzoic acid derivative such as 2-Nitro-4-(methyl sulfonyl) benzoic acid (NMSBA) is an important intermediate for the synthesis of well-known herbicide, Mesotrione.

The substituted benzoic acids are generally synthesized by oxidation of the methyl group by using a wide variety of oxidizing agents such as hydrogen peroxide, air, oxygen, potassium permanganate, alkaline hypochlorite, alkali persulfate, chromium trioxide, and the like. Other methods for the synthesis of the substituted benzoic acids involve oxidation of substituted benzyl alcohol, side chain halogenation of methyl groups, followed by hydrolysis and/or oxidation, depending upon the mono, di or tri halogenation of the methyl groups.

2-Nitro-4-(methyl sulfonyl) benzoic acid (NMSBA) is conventionally synthesized by various methodologies such as NMSBA is prepared by oxidation of 2-nitro-4-methylsulfonyl toluene. NMSBA is also prepared by using 2-nitro-4-methylsulfonyl benzyl chloride or 2-nitro-4-methylsulfonyl benzyl bromide. Further, NMSBA is prepared by reacting 2,4-dinitro benzoic acid with sodium salt of methanesulfinic acid. Still further, NMSBA prepared from 2-nitro-4-methylsulfonyl benzonitrile is also known in the prior art.

However, these methods are associated with drawbacks such as low yield and/or low purity of the oxidized product. Further, the conventional methods involve formation of dinitro impurities in the product which are difficult to remove and hence, these methods involve tedious purification stages, thereby resulting in an expensive process.

There is, therefore, felt a need to provide an alternative process for the preparation of 2-nitro-4-methylsulfonyl benzoic acid, which mitigates the drawbacks mentioned herein above or at least provides a useful alternative.

### OBJECTS

Some of the objects of the present disclosure, which at least one embodiment herein satisfies, are as follows:

It is an object of the present disclosure to ameliorate one or more problems of the prior art or to at least provide a useful alternative.

Another object of the present disclosure is to provide a process for the preparation of 2-nitro-4-methylsulfonyl benzoic acid (NMSBA).

Yet another object of the present disclosure is to provide a simple, economical and environment-friendly process for the preparation of 2-nitro-4-methylsulfonyl benzoic acid (NMSBA).

Still another object of the present disclosure is to provide a process for the preparation of 2-nitro-4-methylsulfonyl benzoic acid with a comparatively high purity and high yield.

Other objects and advantages of the present disclosure will be more apparent from the following description, which is not intended to limit the scope of the present disclosure.

### SUMMARY

The present disclosure relates to a process for the preparation of 2-nitro-4-methylsulfonyl benzoic acid (NMSBA). The process comprises reacting 1-chloro-2-nitro-4-methyl sulfonyl benzene (chloro NMSB) with a compound of Formula II by using a base in a fluid medium at a first predetermined temperature to obtain a compound of Formula III;

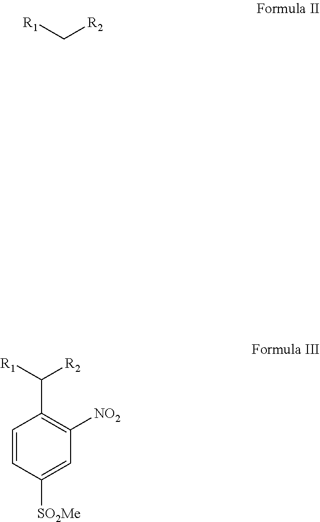

wherein,
R₁ is selected from —CONH₂, —COOR₃, wherein R₃ is selected from C₁-C₄ alkyl; and
R₂ is a cyano group (—CN).

The compound of Formula III is reacted with an oxidizing agent optionally in the presence of a catalyst under stirring to obtain a mixture. Air is optionally passed through the mixture. The mixture is heated at a second predetermined temperature for a predetermined time period to obtain a product mixture comprising 2-Nitro-4-(methyl sulfonyl) benzoic acid.

### DETAILED DESCRIPTION

The present disclosure relates to a process for the preparation of 2-nitro-4-(methylsulfonyl)benzoic acid (NMSBA).

Embodiments are provided so as to thoroughly and fully convey the scope of the present disclosure to the person skilled in the art. Numerous details are set forth, relating to specific components, and methods, to provide a complete understanding of embodiments of the present disclosure. It will be apparent to the person skilled in the art that the details provided in the embodiments should not be construed to limit the scope of the present disclosure. In some embodiments, well-known processes, well-known apparatus structures, and well-known techniques are not described in detail.

The terminology used, in the present disclosure, is only for the purpose of explaining a particular embodiment and such terminology shall not be considered to limit the scope of the present disclosure. As used in the present disclosure, the forms “a,” “an,” and “the” may be intended to include the plural forms as well, unless the context clearly suggests otherwise. The terms “comprises,” “comprising,” “including,” and “having,” are open ended transitional phrases and therefore specify the presence of stated features, integers, steps, operations, elements, modules, units and/or components, but do not forbid the presence or addition of one or more other features, integers, steps, operations, elements, components, and/or groups thereof. The particular order of steps disclosed in the method and process of the present disclosure is not to be construed as necessarily requiring their performance as described or illustrated. It is also to be understood that additional or alternative steps may be employed.

The terms first, second, third, etc., should not be construed to limit the scope of the present disclosure as the aforementioned terms may be only used to distinguish one element, component, region, layer or section from another component, region, layer or section. Terms such as first, second, third etc., when used herein do not imply a specific sequence or order unless clearly suggested by the present disclosure.

Benzoic acids and its substituted derivatives, are vital intermediates for the production of agricultural chemicals. Mesotrione, a well-known herbicide is obtained from the important chemical intermediate i.e., 2-Nitro-4-(methylsulfonyl) benzoic acid (NMSBA) which is synthesized by a wide variety of methodologies. However, these methods are associated with drawbacks such as low yield and/or low purity of the product and, hazardous and tedious process steps.

The present disclosure provides a simple, economical, and environment-friendly process for the preparation of 2-Nitro-4-(methylsulfonyl) benzoic acid (NMSBA).

NMSBA is represented by Formula I:

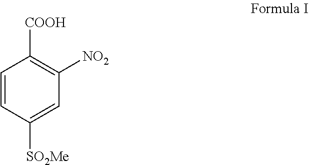 [SMILES: CS(=O)(=O)c1ccc(C(=O)O)c([N+](=O)[O-])c1]

The process for the preparation of 2-nitro-4-(methylsulfonyl)benzoic acid, the process comprises the following steps:
a) reacting 1-chloro-2-nitro-4-methyl sulfonyl benzene (chloro NMSB) with a compound of Formula II by using a base in a fluid medium at a first predetermined temperature to obtain a compound of Formula III;

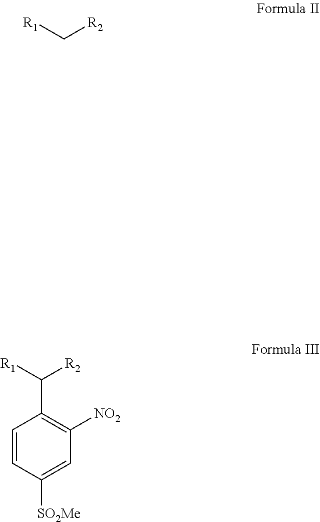

wherein,
R₁ is selected from —CONH₂, and —COOR₃, wherein R₃ is selected from C₁-C₄ alkyl; and
R₂ is a cyano group (—CN);
b) reacting the compound of Formula III with an oxidizing agent optionally in the presence of a catalyst under stirring to obtain a mixture;
c) optionally passing a predetermined amount of air through the mixture; and
d) heating the mixture to a second predetermined temperature for a predetermined time period to obtain a product mixture comprising 2-Nitro-4-(methyl sulfonyl)benzoic acid.

The preparation of 2-Nitro-4-(methylsulfonyl) benzoic acid (NMSBA) (I), in accordance with the process of the present disclosure, is shown in Scheme I as given below:

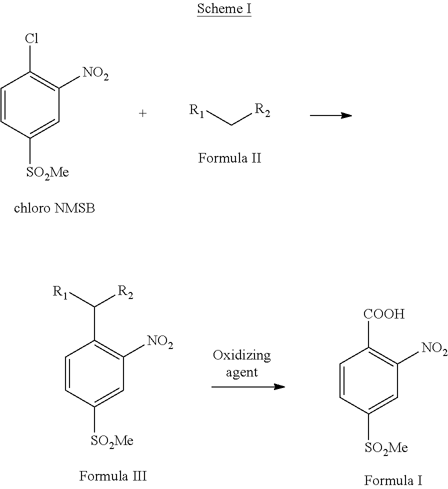

wherein, R₁ is selected from —CONH₂, —COOR₃, wherein R₃ is selected from C₁-C₄ alkyl; and
R₂ is a cyano group (—CN).

The process for preparing 2-Nitro-4-(methylsulfonyl) benzoic acid (NMSBA) is described in detail herein below.

Step-(a): 1-chloro-2-nitro-4-methyl sulfonyl benzene (chloro NMSB) is reacted with a compound of Formula II by using a base in a fluid medium at a first predetermined temperature to obtain a compound of Formula III;

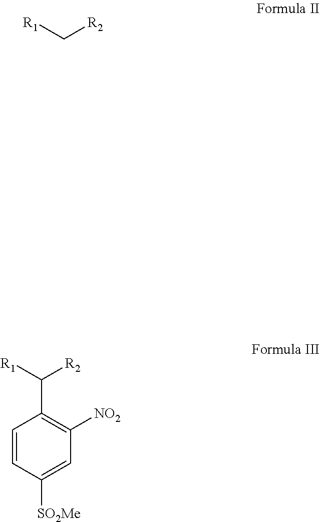

Wherein,
R₁ is selected from —CONH₂, —COOR₃, wherein, R₃ is selected from C₁-C₄ alkyl; and
R₂ is a cyano group (—CN)

In accordance with the present disclosure, the compound of formula II is selected from cyano acetamide and methyl cyano acetate.

In accordance with the present disclosure, the amount of the compound of Formula II is in the range of 1.0 mole to 1.5 moles with respect with the amount of 1-chloro-2-nitro-4-methyl sulfonyl benzene. In an exemplary embodiment, 1.05 moles of the compound of Formula II is used. In another exemplary embodiment, 1 mole of the compound of Formula II is used.

In accordance with the present disclosure, the base is selected from the group consisting of alkali metal hydroxides, alkali metal alkoxide and alkali metal hydrides.

The alkali metal hydroxide is selected from the group consisting of sodium hydroxide, calcium hydroxide and potassium hydroxide.

The alkali metal alkoxide is selected from the group consisting of sodium methoxide, potassium methoxide, sodium ethoxide and potassium ethoxide.

The alkali metal hydride is selected from the group consisting of sodium hydride, potassium hydride, calcium hydride and lithium hydride.

In an exemplary embodiment, the base is sodium hydroxide. In another exemplary embodiment, the base is potassium hydroxide.

The fluid medium is selected from dimethyl sulfoxide (DMSO), dimethyl formamide (DMF) and dimethylacetamide (DMAC). In an exemplary embodiment, the fluid medium is dimethyl sulfoxide (DMSO). In another exemplary embodiment, the fluid medium is dimethyl formamide (DMF).

In accordance with the present disclosure, the compound of formula III is selected from 2-cyano-2-(4-methylsulfonyl)-2-nitro-phenyl acetamide and methyl-2-cyano-2-(4-(methyl sulfonyl)-2-nitrophenyl)acetate. In an exemplary embodiment, the compound of formula III is 2-cyano-2-(4-methylsulfonyl)-2-nitro-phenyl acetamide. In another exemplary embodiment, the compound of formula III is methyl-2-cyano-2-(4-(methyl sulfonyl)-2-nitrophenyl)acetate.

The first predetermined temperature is in the range of 5° C. to 50° ° C. In an exemplary embodiment, the first predetermined temperature is 30° ° C. In another exemplary embodiment, the first predetermined temperature is 10° C.

Step-(b): The compound of Formula III is reacted with an oxidizing agent optionally in the presence of a catalyst under stirring to obtain a mixture.

In accordance with the present disclosure, a concentration of the oxidizing agent is in the range of 5% to 70% and the amount of oxidizing agent is in the range of 2 to 6 moles per mole of the compound of Formula III.

The oxidizing agent is nitric acid (HNO₃).

Other oxidizing agents like H₂SO₄ and NaOCl do not oxidized the compound of Formula III to obtain the desired carboxylic acid, instead it results in other products which does not give carboxylic acid even after purification.

In an exemplary embodiment, the nitric acid used in the reaction has a concentration of 30% and the amount of nitric acid used is 5 moles per mole of the compound of Formula III.

The catalyst is vanadium pentoxide.

In accordance with the present disclosure, the amount of the catalyst is in the range of 1 gram to 12 grams per mole of the compound of Formula III. In an exemplary embodiment, the amount of vanadium pentoxide is 5 grams per mole of the compound of Formula III.

Step-(c): Air is optionally passed through the mixture.

In accordance with the present disclosure, the amount of air passed through the mixture is in the range of 15 to 25 liters per hour per mole of compound of Formula III.

In an exemplary embodiment, the amount of air passed through the mixture is 20 liters per hour per mole of the compound of Formula III.

In another embodiment, air is replaced by nitrogen.

Step-(d): The mixture is heated at a second predetermined temperature for a predetermined time period to obtain a product mixture comprising 2-Nitro-4-(methyl sulfonyl) benzoic acid.

The second predetermined temperature is in the range of 95° C. to 100° C. and the predetermined time period is in the range of 0.5 hour to 5 hours.

In an exemplary embodiment the mixture is heated to 98° C. for 1 hour.

Further, the product mixture so obtained is cooled to a temperature in the range of 20° C. to 60° C. to obtain a residue. In an embodiment the solution is cooled to the temperature of 50° C.

The cooled solution is filtered to separate a residue comprising 2-Nitro-4-(methyl sulfonyl)benzoic acid. The separated residue of 2-Nitro-4-(methyl sulfonyl)benzoic acid is mixed with water to obtain a slurry. The slurry is filtered to obtain a solid and a filtrate. The solid is washed with water twice to obtain 2-Nitro-4-(methyl sulfonyl)benzoic acid.

The filtrate is extracted with an organic solvent followed by distilling the organic solvent to obtain 2-Nitro-4-(methyl sulfonyl)benzoic acid.

The organic solvent is selected from ethyl acetate and butyl acetate.

The yield of 2-Nitro-4-(methyl sulfonyl)benzoic acid so obtained is more than 75% and a purity is greater than 90%.

In an embodiment of the present disclosure, the yield of 2-Nitro-4-(methyl sulfonyl)benzoic acid so obtained is more than 80% and a purity is greater than 93%.

In an exemplary embodiment of the present disclosure, the process for the preparation of 2-Nitro-4-(methylsulfonyl) benzoic acid (NMSBA) (I) comprises the following steps:
a) reacting 1-chloro-2-nitro-4-methyl sulfonyl benzene (chloro NMSB) with methyl cyano acetate (Formula II) by using a base in a fluid medium at a first predetermined temperature to obtain methyl-2-cyano-2-(4-(methyl sulfonyl)-2-nitrophenyl)acetate (Formula III);

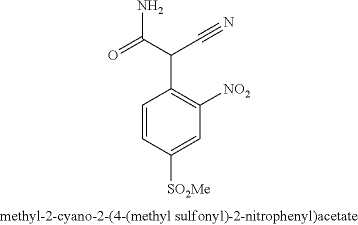 [SMILES: CS(=O)(=O)c1ccc(C(C#N)C(N)=O)c([N+](=O)[O-])c1]

b) reacting methyl-2-cyano-2-(4-(methyl sulfonyl)-2-nitrophenyl)acetate with an oxidizing agent optionally in the presence of a catalyst under stirring to obtain a mixture;
c) optionally passing a predetermined amount of air through the mixture; and
d) heating the mixture to a second predetermined temperature for a predetermined time period to obtain a product mixture comprising 2-Nitro-4-(methyl sulfonyl)benzoic acid.

The process for the preparation of 2-Nitro-4-(methylsulfonyl) benzoic acid (NMSBA) (I), is represented as Scheme Ia given below, wherein Formula II is methyl cyano acetate:

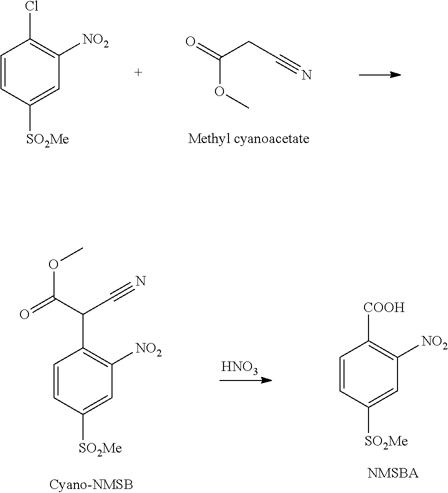

In another exemplary embodiment of the present disclosure, the process for the preparation of 2-Nitro-4-(methylsulfonyl) benzoic acid (NMSBA) (I) 1. comprises the following steps:
a) reacting 1-chloro-2-nitro-4-methyl sulfonyl benzene (chloro NMSB) with cyano acetamide (Formula II) by using a base in a fluid medium at a first predetermined temperature to obtain 2-cyano-2-(4-methylsulfonyl)-2-nitro-phenyl acetamide (Formula III);

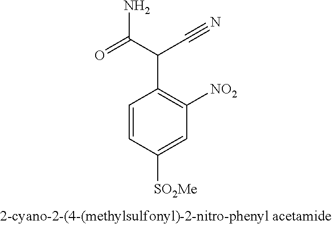 [SMILES: CS(=O)(=O)c1ccc(C(C#N)C(N)=O)c([N+](=O)[O-])c1]

b) reacting methyl-2-cyano-2-(4-(methyl sulfonyl)-2-nitrophenyl)acetate with an oxidizing agent optionally in the presence of a catalyst under stirring to obtain a mixture;
c) optionally passing a predetermined amount of air through the mixture; and
d) heating the mixture to a second predetermined temperature for a predetermined time period to obtain a product mixture comprising 2-Nitro-4-(methyl sulfonyl)benzoic acid.

The process for the preparation of 2-Nitro-4-(methylsulfonyl) benzoic acid (NMSBA) (I), is represented as Scheme Ib given below, wherein Formula II is cyano-acetamide:

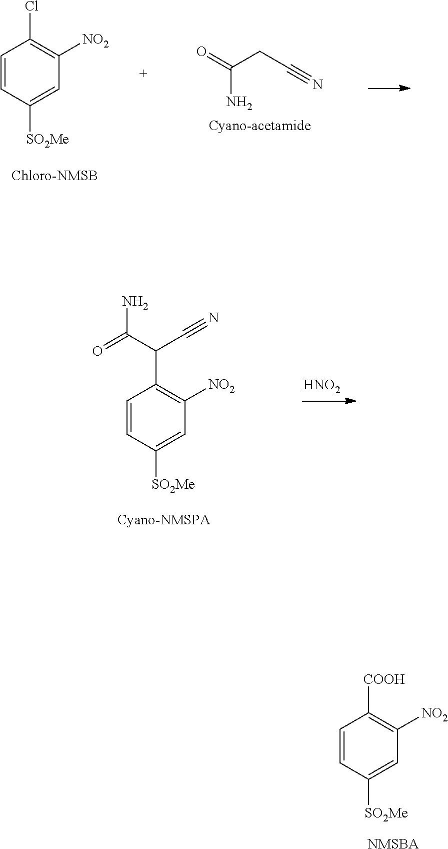

The process of the present disclosure is simple and employs inexpensive and easily available reagents. Thus, the process of the present application is economical.

The process of the present disclosure doesn't require any further purification step. Thus, the process of the present disclosure is simple and environment friendly.

The foregoing description of the embodiments has been provided for purposes of illustration and not intended to limit the scope of the present disclosure. Individual components of a particular embodiment are generally not limited to that particular embodiment, but, are interchangeable. Such variations are not to be regarded as a departure from the present disclosure, and all such modifications are considered to be within the scope of the present disclosure.

The present disclosure is further described in light of the following experiments which are set forth for illustration purpose only and not to be construed for limiting the scope of the disclosure. The following experiments can be scaled up to industrial/commercial scale and the results obtained can be extrapolated to industrial scale.

### EXPERIMENTAL DETAILS

### Experiment 1: Step (a): Preparation of the Compound of Formula III

### Examples 1-2: Preparation of Cyano-NMSB from Chloro-NMSB and Methyl Cyanoacetate

### General Procedure:

A predetermined amount of base was added to a predetermined amount of fluid medium to obtain a mixture. The mixture was cooled to 28° C. to obtain a mass. A predetermined amount of a clear solution of methyl cyanoacetate in a predetermined amount of a fluid medium was added to the mass at 30° C. over a time period of 1 hour to obtain a first reaction mixture. A predetermined amount of a clear solution of Chloro-NMSB in a predetermined amount of a e fluid medium was added to the first reaction mixture at 30° C. over a time period of 2 hours to obtain a second reaction mixture. The second reaction mixture was maintained at 30° C. for 2 hours to obtain a product mass. The product mass was drowned in water and acidified with H₂SO₄ to isolate the compound of Formula III (Cyano-NMSB).

The predetermined amounts of the components used in the examples 1-2 are as summarized in Table 1a.

TABLE 1a
Preparation of Compound of Formula III- Cyano-NMSB [2-
cyano-2-(4-methylsulfonyl)-2-nitro-phenyl acetate]

| | | |
|---|---|---|
| Example no. | 1 | 2 |

| | | | | | |
|---|---|---|---|---|---|
| Reactant | Wt. of Chloro | 902 | g | 58.9 | g |
| | NMSB | | | | |
| Compound of | Wt. of Methyl | 398 | g | 26 | g |
| Formula II | cyano acetate | | | | |
| Base | NaOH | 335 | g | 21.9 | g |

| Fluid medium | DMSO | | DMF | |
|---|---|---|---|---|
| | (3481 ml) | | (250 ml) | |
| H₂SO₄ | 4N | | 5N | |
| | (410 ml) | | (25 ml) | |
| Water | 5000 | ml | 2000 | ml |

| | | | |
|---|---|---|---|
| Compound of | Yield | 98% | 90.5% |
| Formula III | Purity | 99.5% | 98.12% |
| (Cyano-NMSB) | | | |

From Examples 1 and 2, it was observed that when DMSO was used as a fluid medium, the yield of cyano-NMSB was better as compared to the yield when DMF was used. However, in case of DMF, solvent recovery is easy due to lower boiling point of DMF (153° C.) as compared to DMSO (189° C.). Thus, both the fluid media, DMF and DMSO can be advantageously used for the reaction of Chloro NMSB with methyl cyano acetate.

### Examples 3-6: Preparation of Cyano-NMSPA from Chloro-NMSB and Cyano Acetamide

### General Procedure:

A predetermined amount of base was added to a predetermined amount of fluid medium and cooled to a temperature in the range of 3° C. to 18° C. to obtain a mass. A predetermined amount of a clear solution of cyano acetamide in a predetermined amount of a fluid medium was added to the mass at a temperature in the range of 3° C. to 17° C. over a time period of 1 hour to obtain a first reaction mixture. A predetermined amount of a clear solution of Chloro-NMSB in a predetermined amount of a fluid medium was added to the first reaction mixture at a temperature in the range of 5° C. to 22° C. over a time period in the range of 1.25 hours to 2 hours to obtain a second reaction mixture. The second reaction mixture was maintained at a temperature in the range of 5° C. to 22° ° C. for 2 to 3 hours to obtain a product mass. The product mass was drowned in water and acidified with H₂SO₄ to isolate the compound of Formula III (Cyano-NMSPA).

The predetermined amounts of the components used in examples 3-6 are as summarized in Table 1b.

TABLE 1b
Preparation of Compound of Formula III- Cyano-NMSPA [2-
cyano-2-(4-methylsulfonyl)-2-nitro-phenyl acetamide]

| Example No. | | 3 | | 4 | | 5 | | 6 | |
|---|---|---|---|---|---|---|---|---|---|
| Reactant | Wt. of | 59 | g | 59 | g | 117.8 | g | 117.8 | g |
| | Chloro | | | | | | | | |
| | NMSB | | | | | | | | |
| Compound of | Wt. of | 22.15 | g | 22.15 | g | 44.1 | g | 44.1 | g |

| | |
|---|---|
| Formula II | Cyano |
| | acetamide |

| Base | NaOH | | KOH | | NaOH | | KOH | |
|---|---|---|---|---|---|---|---|---|
| | 22 g | | 34.2 g | | 44 g | | 44 g | |
| Fluid medium | | | | | | | | |
| Cooling Temp. | 15 | | 15 | | 10 | | 10 | |
| (° C.) | | | | | | | | |
| Reaction Temp. | 15 | | 15 | | 10 | | 10 | |
| (° C.) | | | | | | | | |
| Reaction Time period | 2 | | 3.5 | | 3 | | 3 | |
| (hour(s)) | | | | | | | | |
| H₂SO₄ | 40% | | 40% | | 40% | | 40% | |
| | (80 ml) | | (86 ml) | | (145 ml) | | (145 ml) | |
| Water | 1000 | ml | 1000 | ml | 2200 | ml | 2200 | ml |

| | | | | | |
|---|---|---|---|---|---|
| Compound of | Yield | 86% | 87.6 | 86% | 89% |
| Formula III | Purity | 95% | 88% | 91% | 92.13% |
| (Cyano-NMSB) | | | | | |

From Examples 3 to 6, it was observed that when DMSO/DMF was used as a fluid medium, the yield of cyano-NMSB was almost same. However, in case of DMF, solvent recovery is easy due to lower boiling point of DMF (153° C.) as compared to DMSO (189° C.). Thus, both the fluid media, i.e. DMF and DMSO can be advantageously used for the reaction of Chloro NMSB with cyano acetamide.

### Experiment 2: Steps (b)-(d): General Procedure for Preparing 2-Nitro-4-(Methyl Sulfonyl) Benzoic Acid (NMSBA) in Accordance with the Process of the Present Disclosure

### General Procedure:

A predetermined amount of Nitric acid was charged to a reactor. A predetermined amount of Vanadium pentoxide was added in the nitric acid in the reactor under stirring to obtain a clear solution. A predetermined amount of Compound of Formula III obtained from step (a) (Example 1-6) was mixed in the clear solution to obtain a mixture. Air was passed through the mixture and the mixture was heated to 98° C. for 1 hour to obtain the product mixture comprising 2-Nitro-4-(methyl sulfonyl)benzoic acid. The progress of the reaction was monitored by HPLC.

Further, the product mixture was cooled to 50° C. to obtain a cooled product mixture. The cooled product mixture was filtered to obtain a residue. The residue was mixed with water to obtain a slurry. The slurry was filtered to obtain a solid and a filtrate. Further, the solid was washed with water twice to obtain 2-Nitro-4-(methyl sulfonyl)benzoic acid. The filtrate was extracted with butyl acetate and the butyl acetate was distilled off from the butyl acetate extract to obtain a second residue (solid) of 2-Nitro-4-(methyl sulfonyl)benzoic acid.

The predetermined amounts of the components used in the reaction of steps (b)-(d) are summarized in Tables 2a and 2b.

TABLE 2a
Process for preparing 2-Nitro-4-(methyl sulfonyl) benzoic
acid (NMSBA) by using Cyano-NMSB (Formula III)

| | Formula | | | | | | | |
|---|---|---|---|---|---|---|---|---|
| | III | | Oxidizing agent | | Catalyst | Air/ | Product | |
| Ex. | Cyano | | HNO₃ | | V₂O₅ | Nitrogen | NMSBA | |
| No. | NMSB | | (strength) | m/m | g/m | 20 lit/h/m | Yield | Purity |
| 1 | 149 | g | 30% | 5 | 5 | Air | 90% | 98.9% |
| 2 | 298 | g | 20% | 3.5 | 10 | Air | 93.1% | 96.5% |
| 3 | 298 | g | 20% | 3.5 | 1 | Air | 89.9% | 93.5% |
| 4 | 14.9 | g | 10% | 5 | 1 | No air | 98% | 85% |
| 5 | 149 | g | 65% | 3.5 | 1 | Air | 94.5% | 91.3% |
| 6 | 29.8 | g | 20% | 5 | Without | No air | 82% | 56.6% |
| | | | | | catalyst | | | |
| 7 | 74.5 | g | 30% | 5 | Without | Air | 70.2% | 91% |
| | | | | | catalyst | | | |
| 8 | 29.8 | g | 20% | 5 | 1 | Nitrogen | 45.7% | 60.7% |
| 9 | 104.5 | g | 40% | 5 | 5 | Air | 89% | 99% |
| 10 | 104.5 | g | 50% | 5 | 5 | Air | 85.9% | 99.2% |
| 11 | 29.8 | g | 20% | 5 | 1 | No air | 93.9% | 88% |

From Table 2a, it was observed that the oxidation reaction by using 20% to 30% HNO₃ and catalyst in an amount in the range of 1 g to 5 g with passing air gave better results in terms of yield and purity. The use of excess amount of catalyst (>5 g) or excess amount of HNO₃ (>30%) would not result in higer yield or higher purity. Thus, the use of optimum amount of HNO₃ as an oxidizing agent along with optimum amount of catalyst and air was found to give high yield and purity as compared to the other examples (examples 6 to 8), wherein the yields and purity were comparatively low as the reaction did not complete in the absence of either air or catalyst or both.

TABLE 2b
Process for preparing 2-Nitro-4-(methyl sulfonyl) benzoic
acid (NMSBA) by using Cyano-NMSPA (formula III)
Preparation of NMSBA from Cyano-NMSPA [2-cyano-2-(4-
methylsulfonyl)-2-nitro-phenyl acetamide]

| | Formula | | | | | | |
|---|---|---|---|---|---|---|---|
| | III | Oxidizing agent | | Catalyst | Air/ | | |
| Ex. | Cyano- | HNO₃ | | V₂O₅ | Nitro- | Product | |
| No. | NMSPA | Strength | m/m | g/m | gen | Yield | Purity |
| 12 | 0.2 m | 30% | 5 | 5 | Air | 75% | 93% |
| 13 | 0.2 m | 20% | 5 | 5 | Air | 97% | 95% |

From Table 2b, it was observed that the oxidation reaction by using 20% HNO₃ and catalyst from 1 g to 5 g with passing air gave better results in terms of yield and purity. The catalyst and air are must for the reaction to give better and high yields whereas, in the absence of the catalyst and air, the reaction does not go to completion.

### Technical Advancements

The present disclosure described herein above has several technical advantages including, but not limited to, the realization of a process for the preparation of 2-nitro-4-(methylsulfonyl)benzoic acid, which:
is simple and environment-friendly;
produces product in high yield and high purity;
is less hazardous reaction involving safety;
involves shorter reaction time and simple work up methodology giving high productivity; and
produces NMSBA devoid of di-nitro impurities and hence avoids carcinogenic impurity in the final product-Mesotrione.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

The use of the expression “at least” or “at least one” suggests the use of one or more elements or ingredients or quantities, as the use may be in the embodiment of the disclosure to achieve one or more of the desired objects or results.

Any discussion of documents, acts, materials, devices, articles or the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values mentioned for the various physical parameters, dimensions or quantities are only approximations and it is envisaged that the values higher/lower than the numerical values assigned to the parameters, dimensions or quantities fall within the scope of the disclosure, unless there is a statement in the specification specific to the contrary.

While considerable emphasis has been placed herein on the components and component parts of the preferred embodiments, it will be appreciated that many embodiments can be made and that many changes can be made in the preferred embodiments without departing from the principles of the disclosure. These and other changes in the preferred embodiment as well as other embodiments of the disclosure will be apparent to those skilled in the art from the disclosure herein, whereby it is to be distinctly understood that the foregoing descriptive matter is to be interpreted merely as illustrative of the disclosure and not as a limitation.

1. A process for the preparation of 2-nitro-4-(methylsulfonyl)benzoic acid (NMSBA), said process comprising the following steps:
a) reacting 1-chloro-2-nitro-4-methyl sulfonyl benzene (chloro NMSB) with a compound of Formula II by using a base in a fluid medium at a first predetermined temperature to obtain a compound of Formula III; 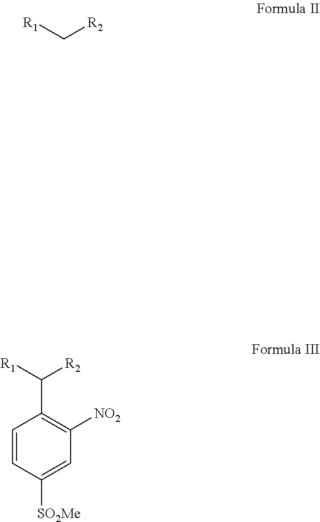
wherein,
R₁ is selected from —CONH₂, and —COOR₃, wherein R₃ is selected from C₁-C₄ alkyl; and
R₂ is a cyano group (—CN);
b) reacting said compound of Formula III with an oxidizing agent optionally in the presence of a catalyst under stirring to obtain a mixture;
c) optionally passing a predetermined amount of air through said mixture; and
d) heating said mixture to a second predetermined temperature for a predetermined time period to obtain a product mixture comprising 2-Nitro-4-(methyl sulfonyl)benzoic acid,
wherein a yield of 2-Nitro-4-(methyl sulfonyl)benzoic acid is greater than 75% and a purity is greater than 90%.

2. The process as claimed in claim 1, wherein an amount of said compound of Formula II is in the range of 1.0 mole to 1.5 moles with respect to the amount of 1-chloro-2-nitro-4-methyl sulfonyl benzene.

3. The process as claimed in claim 1, wherein said compound of Formula II is selected from cyano acetamide and methyl cyano acetate.

4. The process as claimed in claim 1, wherein said base is selected from the group consisting of alkali metal hydroxides, alkali metal alkoxide and alkali metal hydrides.

5. The process as claimed in claim 4, wherein said alkali metal hydroxide is selected from the group consisting of sodium hydroxide, calcium hydroxide and potassium hydroxide; said alkali metal alkoxide is selected from the group consisting of sodium methoxide, potassium methoxide, sodium ethoxide and potassium ethoxide; and said alkali metal hydride is selected from the group consisting of sodium hydride, potassium hydride, calcium hydride and lithium hydride.

6. The process as claimed in claim 1, wherein said fluid medium is selected from the group consisting of dimethyl sulfoxide (DMSO), dimethyl formamide (DMF) and dimethylacetamide (DMAC).

7. The process as claimed in claim 1, wherein said compound of Formula III is selected from 2-cyano-2-(4-methylsulfonyl)-2-nitro-phenyl acetamide and methyl-2-cyano-2-(4-(methyl sulfonyl)-2-nitrophenyl)acetate.

8. The process as claimed in claim 1, wherein said first predetermined temperature is in the range of 5° C. to 50° C.

9. The process as claimed in claim 1, wherein said first predetermined temperature is in the range of 10° C. to 30° C.

10. The process as claimed in claim 1, wherein a concentration of said oxidizing agent is in the range of 5% to 70% and wherein the amount of said oxidizing agent is in the range of 2 moles to 6 moles per mole of said compound of Formula III.

11. The process as claimed in claim 1, wherein a concentration of said oxidizing agent is in the range of 10% to 65% and wherein the amount of said oxidizing agent is in the range of 3 moles to 5 moles per mole of said compound of Formula III.

12. The process as claimed in claim 1, wherein said oxidizing agent is nitric acid (HNO₃).

13. The process as claimed in claim 1, wherein said catalyst is vanadium pentoxide.

14. The process as claimed in claim 1, wherein said predetermined amount of air is in the range of 15 to 25 liters per hour per mole of the compound of Formula III.

15. The process as claimed in claim 1, wherein said predetermined amount of air is 20 liters per hour per mole of the compound of Formula III.

16. The process as claimed in claim 1, wherein said second predetermined temperature is in the range of 95° C. to 100° C. and wherein said predetermined time period is in the range of 0.5 hour to 5 hours.

17. The process as claimed in claim 1, wherein said second predetermined temperature is 98° C. and wherein said predetermined time period is 1 hour.

18. The process as claimed in claim 1, wherein the yield of 2-Nitro-4-(methyl sulfonyl)benzoic acid is greater than 80% and the purity is greater than 93%.

19. The process as claimed in claim 1, wherein said process comprises the following steps:
a) reacting 1-chloro-2-nitro-4-methyl sulfonyl benzene (chloro NMSB) with methyl cyano acetate (Formula II) by using a base in a fluid medium at a first predetermined temperature to obtain methyl-2-cyano-2-(4-(methyl sulfonyl)-2-nitrophenyl)acetate (Formula III); 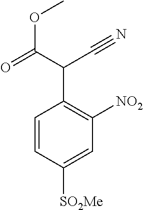 [SMILES: COC(=O)C(C#N)c1ccc(S(C)(=O)=O)cc1[N+](=O)[O-]]
b) reacting methyl-2-cyano-2-(4-(methyl sulfonyl)-2-nitrophenyl)acetate with an oxidizing agent optionally in the presence of a catalyst under stirring to obtain a mixture;
c) optionally passing a predetermined amount of air through said mixture; and
d) heating said mixture to a second predetermined temperature for a predetermined time period to obtain a product mixture comprising 2-Nitro-4-(methyl sulfonyl)benzoic acid.

20. The process as claimed in claim 1, wherein said process comprises the following steps:
a) reacting 1-chloro-2-nitro-4-methyl sulfonyl benzene (chloro NMSB) with cyano acetamide by using a base in a fluid medium at a first predetermined temperature to obtain 2-cyano-2-(4-methylsulfonyl)-2-nitro-phenyl acetamide; 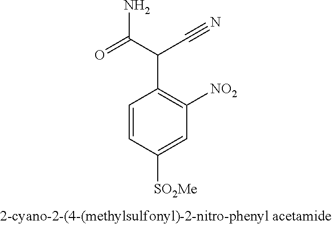 [SMILES: CS(=O)(=O)c1ccc(C(C#N)C(N)=O)c([N+](=O)[O-])c1]
b) reacting methyl-2-cyano-2-(4-(methyl sulfonyl)-2-nitrophenyl)acetate with an oxidizing agent optionally in the presence of a catalyst under stirring to obtain a mixture;
c) optionally passing a predetermined amount of air through said mixture; and
d) heating said mixture to a second predetermined temperature for a predetermined time period to obtain a product mixture comprising 2-Nitro-4-(methyl sulfonyl)benzoic acid.
